(19) United States
(12) Patent Application Publication
(10) Pub. No.: US 2009/0136587 A1
(43) Pub. Date: May 28, 2009

(54) PHARMACEUTICAL COMPOSITION FOR THE TREATMENT OF NERVE DAMAGE COMPRISING BLOOD PLASMA OR SERUM

(75) Inventors: Won Min Yoo, Seoul (KR); Nae Choon Yoo, Seoul (KR); Ki Chang Keum, Seoul (KR); Sang Yup Lee, Daejeon (KR); Gene Lee, Gyeonggi-do (KR)

Correspondence Address: INTELLECTUAL PROPERTY / TECHNOLOGY LAW, PO BOX 14329, RESEARCH TRIANGLE PARK, NC, 27709, US

(73) Assignee: MEDIGENES CO., LTD, Seoul (KR)

(21) Appl. No.: 11/995,901

(22) PCT Filed: Aug. 8, 2006
(86) PCT No.: PCT/KR2006/003116

§ 371 Date: Jul. 14, 2008

(30) Foreign Application Priority Data
Aug. 11, 2005 (KR) 10-2005-0073653
Aug. 11, 2005 (KR) 10-2005-0073654

(51) Int. Cl. A61K 35/16

(52) U.S. Cl. 424/530

(57) ABSTRACT

The present invention relates to a pharmaceutical composition for the treatment of nerve damage, and more particularly to a pharmaceutical composition for the treatment of nerve damage, which contains blood plasma or serum as an active ingredient. The inventive composition regenerates nerve cells after spinal nerve damage and provides complete structural continuity in the spinal nerve lesion sites. Thus, the composition is useful for the treatment of nerve damage.

### TECHNICAL FIELD

The present invention relates to a pharmaceutical composition for the treatment of nerve damage, and more particularly to a pharmaceutical composition for the treatment of nerve damage, which contains a pharmaceutically effective amount of blood plasma or serum as an active ingredient.

### BACKGROUND ART

The nervous system is divided into the peripheral nervous system and the central nervous system, and the peripheral nervous system includes all the nerves except for the brain and spinal cord which is the central nervous system. Also, the nervous system is divided into the somatic nervous system and the autonomic nervous system, in which the somatic nervous system includes the cranial and spinal nerves, and the autonomic nervous system includes the sympathetic and parasympathetic nerves. A single nerve cell consists of cell body, dendrites, and an axon, in which the dendrites serve to transmit a nerve impulse to the cell body, and the axon leads the nerve impulse away from the cell body. To the end of the axon, several synaptic terminals are connected, to which other nerve cells or target cells are connected. The axon comprises a myelin sheath surrounded by a Schwann cell, and a node of Ranvier.

Typical treatment of neurological diseases associated with nerve cell death or damage is to promote nerve regeneration by promoting neurite outgrowth, or to inhibit nerve cell death. Nerve regeneration promoters, which have been known till now, include nerve growth factor (NGF; Levi-Montalcini R. and Hamburger V., J. Exp. Zool., 123:233, 1953), brain-derived neurotrophic factor (BDNF; Leibrock et al., Nature, 341:149, 1989), neurotrophic factor-3 (NT-3; Maisonpierre et. al., Science, 247:1446, 1990), ciliary neurotrophic factor (CNTF; Lin et al., Science, 246:1023, 1990), insulin and insulin-like growth factor (Aizenman et al., Brain Res., 406:32, 1987; Bothwell, J. Neurosci. Res., 8:225, 1982; Recio-Pinto et al., J. Neurosci., 6:1211, 1986; Near et al., PNAS, 89:11716, 1992; Shemer et al., J. Biol. Chem., 262:7693, 1987; Anderson et al., Acta Physiol. Scand., 132:167, 1988; Kanje et al., Brain Res., 475:254, 1988; Sjoberg and Kanje, Brain Res., 485:102, 1989; Nachemson et al., Growth Factors, 3:309, 1990; Re-cio-Pento et al., J. Neurosci. Res., 19:312, 1988; Matteson et al., J. Cell Biol., 102:1949, 1986; Edbladh et al., Brain Res., 641:76, 1994), activin (Schubert et al., Nature, 344:868, 1990), purpurin (Berman et al., Cell, 51:135, 1987), fibroblast growth factor (D. Gospodarowicz et al., Cell Differ., 19:1, 1986; Baird, A. and Bohlen, P., Fibroblast growth factors. In: Peptide growth factors and their receptors 1. (eds. Sporn, M. B. and Roberts, A. B.) 369-418. Spring-Verlag, Berlin, Heidelberg, 1990; and Walter, M. A. et al., Lymphokine Cytokine Res., 12:135, 1993), estrogen (Toran-Allerand et al., J. Steroid Biochem. Mol. Biol., 56:169, 1996; McEwen, B. S. et al., Brain Res. Dev. Brain. Res., 87:91, 1995), platelet-derived growth factors (PDGF; U.S. Pat. No. 6,506,727), fibrin degrading agents (e.g., tissue plasminogen activators), defibrinogenating agents (e.g., ancrod, urokinase, streptokinase or an anticonvulsant; US Patent Publication 2003/0219431 A1) and the like. Substances known to inhibit the death of nerve cells include thrombomodulin analogs (US Patent Publication 2004/0002446 A1).

It is well known that the central nerves of the brain and the spinal cord have no regeneration ability. For this reason, there is no method capable of treating trauma such as spinal cord injury, or nerval regression diseases such as Alzheimer's disease and Parkinson's disease.

Although some studies reported that the nerve cells of the spinal cord show regeneration ability, these studies are incomplete, because the regeneration reaction is short and an axon dies. The reason for this immature reaction is not yet found.

There is a limitation in treating nerve damage using the known nerve regeneration-promoting substances as described above, and several limited therapeutic methods are used for the treatment of spinal nerve damage. Till the latest date, drug therapy following spinal nerve damage comprises intravenously administering a high dosage of steroid methylprednisolone at an early stage of the spinal nerve damage in order to minimize secondary damage (Hall, E. D., Adv. Neurol., 59:241, 1993; Bracken, M. B., J. Neurosurg., 93:175, 2000; Bracken, M. B., Cochrane Database Syst. Rev., 2, 2000; Koszdin, et al., Anesthesiology, 92:156, 2000). However, recent analytical data suggest that this drug therapy should be prohibited (Hurlbert, R. J., J. Neurosurg., 93:1, 2000; Pointillart et al., Spinal Cord, 38:71, 2000; Lankhorst et al., Brain Res., 859:334, 2000), and other drugs still remain in experimental stages.

In recent years, there have been a considerable advancement in understanding factors restricting spinal nerve regeneration and developing strategies for the successful regeneration of the spinal nerve on the basis of this understanding (Ramer, M. S. et al., J. Neurosci. 21:2651, 2001). As a result of such studies, Nogo was suggested as one of inhibitors of central nerve regeneration (Chen, M. S. et al., Nature, 403:434, 2000). However, because the inhibition of Nogo results in regeneration of only part of the axon, other regeneration inhibitors can exist. The regeneration inhibitor candidates can be exemplified by MAG (myelin-associated glycoprotein), tenascin, gangliosides, ephrin, netrin, semaphorins and the like. Although such substances clearly perform an important role in the successful regeneration of the spinal nerve, other requirements should also be considered in the regeneration of the spinal nerve. Examples of these requirements include maximizing positive support for the generation of axons, optimizing the function of living axons in partial lesions, guiding extended regrowth, setting appropriate continuity, and minimizing harmful effects, including initial trauma, inflammation and wound formation. Regarding this, Priestley et al. suggested that the regeneration of injured spinal nerve can be stimulated with fibronectin mats together with neutrophin (Priestley, J. V. et al., Journal of Physiology—Paris 96, 123-133, 2002).

However, the regeneration of nerves by the above-described growth factors has problems in that it is effective locally at certain parts of nerve cells during the regeneration process of nerve cells, and it is not economical, because it costs a great deal of money for the production and purification of the growth factors.

Thus, the present inventors have made extensive efforts to develop a more effective nerve regenerator and, as a result, found that, when a rat model having damaged spinal nerve was treated with mammalian blood plasma or serum, the nerve cells were then regenerated, and the lesion site having the regenerated nerves had a complete structural continuity with other sites, thereby completing the present invention.

### SUMMARY OF THE INVENTION

It is an object of the present invention to provide a pharmaceutical composition for the treatment of nerve damage, which contains a pharmaceutically effective amount of blood plasma or serum as an active ingredient.

To achieve the above object, the present invention provides a pharmaceutical composition for the treatment of nerve damage, which contains a pharmaceutically effective amount of blood plasma or serum as an active ingredient.

The composition according to the present invention is preferably in the form of any one selected from the group consisting of paste, solution, suspension, and gel for local administration. Also, the content of said blood plasma or serum is preferably 0.1-99.9 wt % based on the weight of the composition.

In the present invention, the nerve damage is preferably peripheral nerve damage or spinal damage. Also, the nerve damage is preferably damage in which the nerves are severed. The damage, in which the nerves are severed, is preferably damage in which the length of severed neurites are longer than 10 mm.

In the present invention, the peripheral nerve damage is preferably a nerve disorder associated with a disease selected from the group consisting of diabetic neuropathy, acromegaly, hypothyroidism, AIDS, leprosy, Lyme disease, systemic lupus erythematosus, rheumatoid arthritis, Sjogren's syndrome, periarteritis nodosa, Wegener's granulomatosis, cranial arteritis and sarcoidosis.

Other features and examples of the invention are clarified by the minute descriptions and attached claims as follows.

### BRIEF DESCRIPTION OF DRAWINGS

FIG. 1 shows the results of BBB test for a rat model having damaged nerve cells.

FIG. 2 shows the results of grid walk test for a rat model having damaged nerve cells.

FIG. 3 illustrates photographs showing the results of footprint analysis for rat models (A: normal group; B: control group; and C: treated group).

FIG. 4 shows the results of electrophysiological sensory latency test for a rat model having damaged nerve cells.

FIG. 5 shows histological photographs of lesion sites of a control rat group and a normal rat group.

### DETAILED DESCRIPTION OF THE INVENTION AND PREFERRED EMBODIMENTS THEREOF

As used herein, the term “spinal nerve damage” means all damages occurring when external force is applied to the spinal cord. The spinal nerve damage consists of primary damage and secondary damage in pathophysiologic terms. The primary damage to spinal nerves refers to tissue damage caused by physical rupture upon the occurrence of impact. The primary damage in the human body occurs due to mechanical forces such as impact, compression, traction and laceration, incised wound, and so on. As the most frequent mechanism, the primary damage occurs due to impact together with continuous compression and is mainly attributable to compression fracture, fracture-dislocation, bullet wound and disk rupture.

The secondary damage to spinal nerves is initiated by biochemical mediators released from tissue upon the primary damage. Biochemical substances resulting from the secondary damage cause a pathophysiologic signaling process to gradually damage tissue, thus resulting in cell death. This liberates other biochemical substances, so that a vicious circle of tissue breakdown is continued. Such secondary pathophysiologic processes include various biochemical changes which cause cell membrane damage, blood vessel damage, inflammation, electrolyte imbalance, altered energy metabolism, and oxidative stress.

The degree of nerve injury can be assessed by various methods. Examples of these methods include the Frankel classification system, the ASIA (American Spinal Injury Association) classification system, the Yale classification system, the motor index scale, the modified Barthel index (Wells, J. D. and Nicosia, S., J. Spinal Cord Med., 18:33, 1994) and the like. Patients can be discriminated between complete or incomplete spinal injury. The complete spinal cord damage can be considered to be the case in which muscular strength and sensation are completely lost within 24 hours after traumatic injury, or the anal sphincters are contracted, or sacral sensation is lost and bulbocavernous reflex is lost. The incomplete spinal cord damage refers to the state in which, below injured sites, at least some of the movement or sensation function remains. The incomplete spinal damages are classified into anterior cord syndrome, posterior cord syndrome, central cord syndrome, lateral cord syndrome, nerve root syndrome and the like.

Animal models of making traumatic spinal cord injury have been advantageously used to establish pathophysiologic mechanisms and assess therapeutic effects (Anderson, T. E. and Strokes, B. T., J. Neurotrauma, 9:S135, 1992; Blight, A. R., Central Nervous System Trauma, 2:299, 1985; Blight, A. R. et al., McGraw Hill: New York, 1367-1379, 1966). Such animal models include weight drop model (Kuhn, P. L. and Wrathall, J. R., J. Neurotrauma, 15:125, 1998), crush injury model (Bunge, R. P. et al., Advances in Neurology. FJ Seil (ed), Raven Press: New York, 75-89, 1993; Bunge, R. P. et al., Neuronal Regeneration, Reorganization, and Repair. FJ Seil (ed), Leppincott-Raven Publishers: Philadelphia, 305-315, 1997), contusion injury model (Stokes, B. T. et al., J Neurotrauma, 9:187, 1992) and the like. The contusion injury model is most suitable for imitating a rapid non-perforated traumatic injury. The formed injury can be assessed by tissue examination (e.g., light or electron microscopy, staining and tracing; Gruner, J. A., J. Neurotrauma, 9:123, 1992), measurement of electrophysiological results (e.g., evoked potential; Metz et al., J. Neurotrauma, 17:1, 2000) or behavior evaluation (e.g., outdoor walking ability or postural stability on inclined planes; Basso et al., J. Neurotrauma, 13:343, 1996).

In another aspect, the present invention relates to a pharmaceutical composition for the treatment of peripheral nerve damage in which neurites are severed, particularly the length of severed neurites is more than a few millimeters (e.g., more than 10 mm). The regenerative property of neurons in the peripheral nervous system has limited ability to restore function of a damaged neural pathway. That is the new axons extend randomly, and are often in the wrong direction to be in contact with inappropriate targets and thus can cause abnormal function. For example, if a motor nerve is damaged, regrowing axons may be brought into wrong muscles, thus resulting in paralysis. In addition, in the case where the length of severed neuritis are more than a few millimeters (e.g., more than 10 mm), appropriate nerve regeneration does not occur, either because the neurites fail to grow as long as they need, or because of axonal growth in the wrong direction.

Efforts to repair peripheral nerve damage by surgical means may result in various results. In some cases, the suturing steps used to obtain proper alignment of severed nerve ends stimulates the formulation of scar tissue which is thought to inhibit axon regeneration. Even in the case where scar tissue formation has been reduced, successful nerve regeneration is still limited to nerve damage of less than 10 mm. In addition, the reparative ability of peripheral neurons is significantly inhibited in the case where an injury or neuropathy affects the cell body of nerve cell itself or results in extensive degeneration of a distal axon. The inventive composition regenerates nerve cells and provides complete structural continuity in nerve lesion sites, and thus can treat mammalian peripheral nerve damage in which the length of severed neurites are more than 10 mm.

In still another aspect, the present invention relates to a pharmaceutical composition for treating disease-induced peripheral nerve neuropathies and conditions associated with the diseases. The disease-induced peripheral nerve disorders may be diabetic neuropathies. The diabetic neuropathies can be defined as clinically evident diseases occurring due to symptoms of diabetes without other causes by peripheral nerve disorders. Such neuropathies include symptoms occurring in the autonomic and somatic nervous systems of peripheral nerve system. The diabetic neuropathies damage the nerves just below the skin to cause at least one of the following symptoms: the numbness and tingling in fingers, hands, toes and feet; the impotence in hands and feet; and pain and burning in hands and feet. Also, the disease-induced peripheral neuropathies may be neuropathies associated with acromegaly, hypothyroidism, AIDS, leprosy, Lyme disease, systemic lupus erythematosus, rheumatoid arthritis, Sjogren's syndrome, periarteritis nodosa, Wegener's granulomatosis, cranial arteritis and sarcoidosis.

The inventive composition regenerates nerve cells and provides complete structural continuity in nerve lesions, and thus can treat the above disease-induced peripheral neuropathies and related conditions.

Blood plasma used as an active ingredient in the present invention typically refers to a fluid substance in mammalian blood, namely, a straw-colored liquid substance from which cells and cell fragments are separated and the substance and compositon thereof is well known through literatures in the field (Westerman, P., Plasma Proteins, VII-1 to VII-13, 2002; Wendy, Y. C. et al., Plasma Proteins Pocket Guide, Foundation for Blood Research the whole contents of these literatures will be cited in this application as a reference).

Blood serum used as an active ingredient in the composition of the present invention generally refers to a site where fibrinogen, clotting factors, etc. are removed from blood plasma.

Blood plasma or serum used as an active ingredient in the composition of the present invention include blood plasma or serum isolated from blood of all species of mammals including humans non-human primates, for example, livestock, such as sheep, goats, pigs, horses, dogs and cattles, primates, rodents, etc.

Blood plasma or serum used in the present invention can be readily isolated from blood using conventional methods, such as centrifugation, sedimentation or filtration. Centrifugation can be carried out under suitable condition to precipitate blood cell from blood plasma. For example, centrifuging blood at about 1,400 rpm for 10 minutes is sufficient to precipitate all cell fragments containing platelets as well as red and white blood cells. Supernatant containing plasma can be easily separated from the precipitated cells by standard techniques.

Such filtration can be performed by passing blood through a filter suitable to isolate blood cells from blood plasma. The filter is preferably a microporous membrane capable of passing proteins through it easily.

In addition to fresh liquid plasma preparation or a liquid preparation form obtained by centrifuging or sedimentating after blood was withdrawn, preservation methods in various forms before blood plasma or serum are used, are known, for example, fresh-frozen preparation, cryoprecipitate preparation, a lyophilized preparation or a concentrated preparation. In the present invention, all forms of plasma or serum described above can be used.

Fresh-frozen plasma is prepared by centrifuging blood, which is within 6 hours after withdrawing the blood sample, at about 1,400 rpm for 15 minutes to isolate blood cells and plasma and freezing at about −40□ to −18□. It is preferable to use the Fresh-frozen plasma after thawing it at about 30˜37□ of warm water.

Cryoprecipitated plasma is obtained by isolating white precipitate (cold precipitated protein) (containing many factors, such as VIII:C, fibrinogen, XIII and fibronectin) which is generated when one unit of fresh-frozen plasma is thawed at about 4□, and refreezing it at about −40□ to −18□. For its use, cryoprecipitate preparation is thawed out by leaving it to stand in a refrigerator (1˜6□) overnight or thawed in a water bath (about 4□ for quick use).

Concentrated plasma can be used by isolating plasma from blood, concentrating after mixing the isolated plasma with a concentrating agent, such as dextranomer, SEPHDEX, dextramine, polyacryl amide, BIO-GEL P, silica gel, zeolite, DEBRISAN, crosslinked agarose, starch and alginate gel and isolating the concentrating agent from the concentrated plasma.

In one embodiment of the present invention, blood plasma or serum which can be purchased from Blood Bank can be used. For example, powdered preparations purchased from Blood Bank, liquid preparation of Invitrogen Corporation (for example, Gibco™ Chicken Serum, Gibco™ Goat Serum, Gibco™ Lamb Serum, Gibco™ Porcine Serum, Gibco™ Rabbit Serum) or serum preparation of GeminiBio-Products (USA) (for example, Chicken Serum (Cat.#100-161), Dog Serum (Cat.#100-160), Donor Donkey Serum (Cat.#100-151), Donor Goat Serum (Cat.#100-109), Donor Rat Serum (Cat.#100-155), Feline Serum (Cat.100-153), Guinea Pig Serum (Cat.#100-130), Monkey Serum (Cat.#100-154), Mouse Serum (Cat.#100-113), Porcine Serum (Cat.#100-115), Rabbit Serum (Cat.#100-116), Rat Serum (Cat.#100-150) or Sheep Serum (Cat.#100-117) can be used. These preparations are prepared from blood plasma unit deried from mammals including human and confirmed from the test results that they are non-reactive with antibodies against hepatitis B surface antigen (HBsAg) and hepatitis C(HCV) and negative for antibodies against HIV-1 and HIV-2. All units of blood plasma used to prepare such preparations are certified free of pathogens.

When blood plasma or serum except said preparations are used, it is preferable to inactivate enveloped viruses, such as HIV, hepatitis B and HCV in blood plasma or serum to reduce the potential risk of transmission of infectious agents. The common methods among methods for inactivating blood plasma include pasteurization, dry heat treatment, vapor treatment, organic solvent/detergent mixture treatment (for example, tri(n-butyl)/phosphate/polysorbate 80), low pH (pH 4), cold ethanol fractionation, chromatography, nanofiltration. Recently, UV irradiation, γ-ray irradiation, iodine treatment is being developed. It is preferable to use after blood plasma unit is subjected to continuous cycle of γ-ray irradiation, methylene blue treatment and vapor treatment to inactivate viruses which may exist in blood plasma.

As plasma or serum fractions used in the present invention, plasma or serum fractions, which are powdered by heating, lyophilization or other suitable drying techniques, can be used. For example, blood plasma or serum can be used after freeze-drying at less than −40□ for several days (e.g., about 7 days) to powderize.

To effectively treat nerve damage, the blood plasma or serum used in the inventive pharmaceutical composition should easily act on target sites. For this purpose, the composition suitable for use according to the present invention is prepared in the form of a preparation in which blood plasma or serum as an active ingredient is combined with one or more pharmaceutically acceptable excipients or carriers. As used herein, the term “pharmaceutically acceptable” means the carriers or excipients are miscible with the active ingredient and not harmful to subjects. In the present invention, the blood plasma or serum can be present in an amount of 0.1-99.9 wt % based on the total weight of the pharmaceutical composition. Alternatively, the blood plasma or serum can be used alone, not in combination with excipients, carriers or diluents.

The blood plasma or serum according to the present invention is locally administered. Formulations suitable for local administration include a semi-solid phase, semi-liquid phase or liquid phase formulation, such as paste, gel, solution, emulsion or suspension. A preferred administration route of the inventive composition may be intramuscular, subcutaneous or transdermal injection or infusion. Alternatively, the formulation may be freeze-dried powder, which can be used after being reconstructed with a medium, which is isotonic with physiological body fluid and buffered at suitable pHs. These formulations can be prepared by mixing various components and dissolving or kneading the mixture using any apparatus or method well known in the pharmaceutical field [Remington's Pharmaceutical Science, 18th Edition, 1990, Mack Publishing Company, Easton, Pa. 18042 (Chapter 87: Blaug, Seymour)]. Preferably, the active ingredient can be formulated into paste.

The inventive formulation may contain at least one additional compound selected from the group consisting of: viscosity adjusting agents, such as beeswax, glyceryl tribenenate and glyceryl trimyristate; buffers, such as potassium metaphosphate, potassium phosphate, monobasic sodium acetate, and anhydrous sodium citrate; surfactants such as fatty acid alkali metal, dimethyl dialkyl ammonium halide, alkyl pyridinium halide, alkyl sulfonate, fatty amine oxide, and 2-alkylimidazoline quaternary ammonium; thickeners, such as paraffin, silicon oxide, cetostearyl alcohol, and wax; diluents, such as mannitol, sorbitol, starch, kaolin and sucrose; preservatives, such as paraben; alkalifying agents, such as ammonium carbonate, diethanolamine, monoethanolamine, potassium hydroxide, sodium carbonate, sodium bicarbonate, and sodium hydroxide; acidifying agents, such as hydrochloric acid, nitric acid, acetic acid, amino acid, and citric acid; antioxidants, such as ascorbic acid, sodium ascorbate, butylated hydroxyanisole, butylated hydroxytoluene, monothioglycerol, and sodium formaldehyde sulfoxylate; emulsifying or suspending agents, such as PVP, gelatin, natural sugar, acacia, guar gum, agar, bentonite, sodium carboxymethylcellulose, and polyethylene glycol; stabilizers; and coloring agents, such as iron oxide, titanium dioxide and aluminum lake.

The dosage of the blood plasma or serum according to the present invention should be suitably determined considering patient's health condition and the degree of nerve damage, etc. For adults, it is preferably applied to lesions at a dosage of about 0.0001 to 5 mg/cm² each time.

### EXAMPLES

Hereinafter, the present invention will be described in further detail with reference to examples. It is to be understood, however, that these examples are for illustrative purposes only and are not to be construed to limit the scope of the present invention.

### Example 1

### Preparation of Blood Plasma-Containing Paste

Human-derived blood components (the Central Blood Center, Korea; freshly frozen blood plasma) subjected to HIV, HCV and HBV examinations were thawed at 30° C., and then mixed with physiological saline solution at a ratio of 10 g/5 cc, thus preparing blood plasma-containing paste.

### Example 2

### Preparation of Rat Model Having Damaged Vertebra

Female rats (Sprague-Dawley, about 2-month-old; weighed 200-220 g; distributed from Medical Animal Experimental Department, Yonsei University, Korea) were used in tests. These rats were maintained in a 12-hr light/12-hr dark cycle and permitted free access to feed and water, while these were bred according to Institutional Animal Care and Use Committee Guidelines of Association for Assessment and Accreditation of Laboratory Animal Care International. Starting from 14 days before surgical operations, the animals underwent basic walking training for the BBB test and the grid walk test. At 3 days before surgical operations, the animals were subjected to basic evaluation with respect to their behaviors and movement functions, and thus 25 rats suitable for use in the tests were selected.

The selected rats were completely anesthetized with 2 kg/ml of a mixture of 25 mg/ml of ketamine and 1.3 mg/ml of Rompun and subjected to L2 Ventral Laminectomy, according to the method of Schucht, P. et al., (Experimental Neurology, 176:143, 2002). For the prevention of infection during and after the surgical operation, the animals were intramuscularly injected with antibiotic Cefalexin in an amount of 5 mg/100 g bodyweight/day. The second lumbar vertebra of each of the rats was opened, and a small hole (1 mm²) was punctured in the outside of the left arcus vertebra of the rats using a microrongeur. The blade of a blade holder was inserted into the hole and knifed via the dura mater to the outside of the right arcus vertebra, thus making a traumatic damage at the abdominal portion of the spine. The dorsal musculature of the damaged spinal nerve portion was sutured and ligated with surgical clips. After the surgical operation, the rats were placed on warm sawdust to maintain the body temperature thereof, and the portion below the abdominal region thereof was massaged 3-4 times every day for 7 days so as to discharge the content of the bladder, until the autonomic bladder control thereof was completely restored.

At 3 days after the surgical operation for nerve damage, the BBB test (Basso, D. M., Beattie, M. S. and Bresnahan, J. C., J. Neurotrauma, 12:1, 1995) was carried out to assess the open-field walking ability of the rats. The rats were placed in a transparent flexy glass box having a rough surface and were observed for 4-5 minutes. The animals were rated according to criteria, including joint motion, weight bearing, foreleg hind leg coordination, and tail position, in a scale of 0-21, in which 0: the hind leg has no ability to move, and 21: the animal has normal walking ability.

Also, at 3 days after the surgical operation for spinal nerve damage, the grid walk test (Z'Graggen et al., J. Neurosci., 18; 4744, 1998) was conducted to assess the movement function of the rats. In the grid walk test, the rats were allowed to walk 1-m-long parallel metal rods in order to assess the ability to maintain a regular step cycle. Also, the rats were allowed to go down inclined rods in order to assess the control ability of the hind leg. The assessments were performed by counting the number of missteps. 10 missteps indicate that the rat could not regularly walk and not control the limb. 0-1 misstep indicates that the rat was not damaged and had normal motion ability.

Among the 25 animals, 20 animals showed similar scores in the BBB test and the grid walk test, and the average scores thereof were about 11 in the BBB test and about 6 in the grid walk test. The scores were determined by two observers according a double-blind method. The remaining five animals showed significantly different actions and significantly deviated from the average scores, and thus were excluded from the test animals.

### Example 3

### Treatment of Vertebra-Damaged Rat Model with Blood Plasma-Containing Composition

Among the 20 animals selected in Example 2, 10 animals (a treatment group) were treated in the following manner. The spinal nerve damage portion of the second lumbar vertebra of the 10 animals was applied with the blood plasma-containing paste prepared in Example 1. Then, the dorsal musculature was sutured and the skin was ligated with surgical clip. Meanwhile, the remaining 10 animals (a control group) were treated with physiological saline solution in place of the paste and sutured in the same manner as described above.

### Example 4

### BBB test and grid walk test

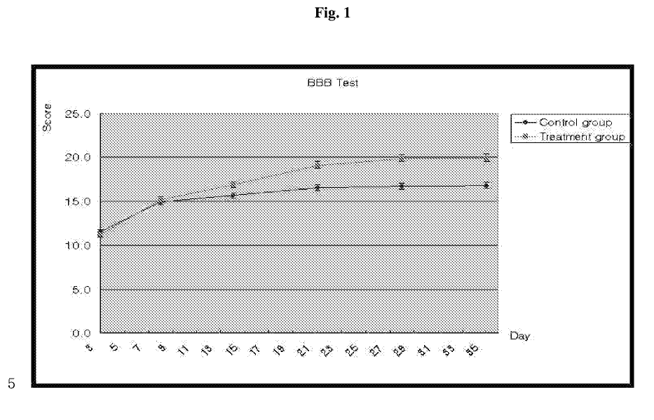

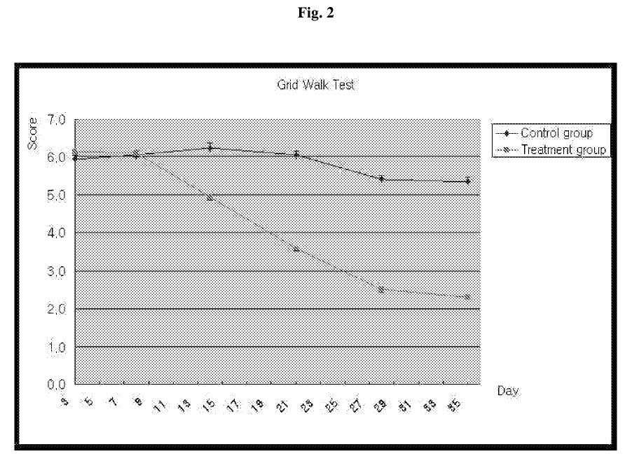

On the 10 animals (the treatment group) treated with 100 μl (2 mg/μl) of the blood plasma-containing paste at the spinal nerve damage portion thereof in Example 3, and on the 10 animals (the control group) treated with physiological saline solution at the nerve damage portion thereof in Example 3, the BBB test and the grid walk test were carried out starting from 3 days after the surgical operation for spinal nerve damage at one-week intervals according to the method described in Example 2. FIG. 1 shows the results of the BBB test, and FIG. 2 shows the results of the grid walk test. As can be seen from the test results, the treatment group and the control group reached the peak of recovery at 35 days after the surgical operation. In addition, as can be seen from two-sample Mamm-Whitney U statistical analysis, the treatment group was recovered in the BBB test (p<0.05) and the grid walk test (p<0.05) while showing significant differences from the control group.

### Example 5

### Footprint Analysis

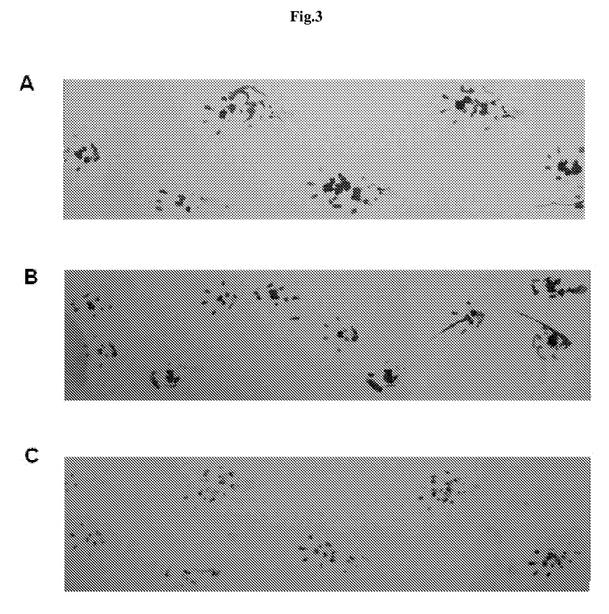

At 35 days after the surgical operation for spinal nerve damage, the foot soles of the treatment group, the control group and normal rats were stained with ink and allowed to walk on white paper. Then, footprint analysis for each of the animal groups was conducted according to criteria, including stride length (distance between foot soles by two continuous steps), the base of support (distance between left and right foot soles), and the angle of rotation (cross-angle between lines set by the angles of the foot sole and the toes (Kunkel-Bagden, E. et al., Exp Neurol., 119, 153, 1993). FIG. 3 shows footprint analysis photographs of the normal group, the control group and the treatment group. As can be seen in FIG. 3, the treatment group showed footprints similar to those of the normal group.

### Example 6

### Electrophysiological Analysis

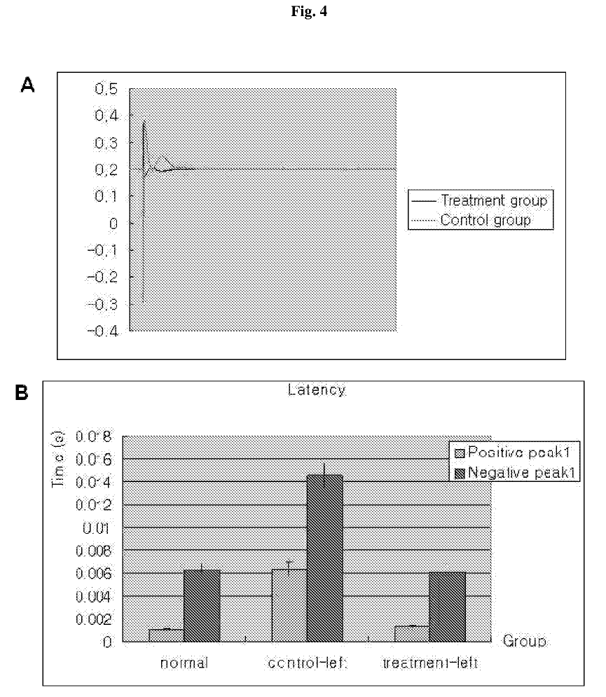

At 35 days after the surgical operation for spinal nerve damage, the control group and treatment group rats were anesthetized with urethane at a dosage of 1.5 g/kg bodyweight, and then the sensory motor cortex and sciatic nerve of hind leg thereof were exposed. A 0.5 mm concentric needle electrode was inserted into the right sensory motor cortex to a depth of 1 mm at about 1-2 mm left of the bregma and about 1 mm rostrocaudal from the bregma, and was applied with an electrical impulse of 3 mA to find out a position of transmitting an optimal signal to the sciatic nerve of the hind leg. The response intensity and sensory nerve latency at this optimal stimulation position were analyzed off-line. FIG. 4 shows the results of sensory nerve latency by a two-independent-sample t-test. As can be seen in the results shown in FIG. 4, the average latency of the treatment group for the sciatic nerve was significantly lower than the average latency of the control group (p<0.05). This indicates the blood plasma-containing paste regenerates nerve cells, and axons are connected by the regenerated nerve cells, thus performing a normal function.

### Example 7

### Histological Analysis

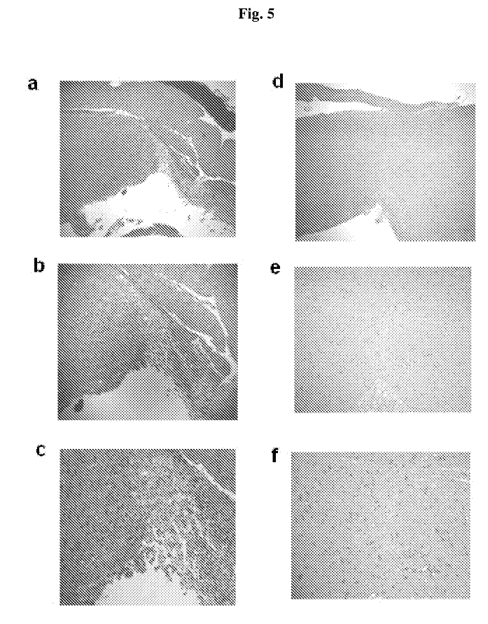

At 35 days after the surgical operation for spinal serve damage, the control group rats and the treatment group rats were humanely sacrificed by injecting an excessive amount of an anesthetic. The second lumbar vertebra of the spinal nerve damage portion, and the thoracic vertebra, third and fourth lumbar vertebrae connected thereto, were removed, and then the remaining tissue was immersed in 10% buffer formalin for 48 hours and embedded in paraffin. The tissue was longitudinally sectioned in a thickness of 2-5 μm in the sagittal plane to prepare tissue segments which were then continuously placed on a microscope slide. The slide was stained with hematoxylin and eosin. FIGS. 5A to 5C are enlarged photographs (5-fold, 10-fold and 20-fold magnifications) of the tissue in the control group, and FIGS. 5D to 5F are enlarged photographs (5-fold, 10-fold and 20-fold magnifications) of the treatment group tissue. As shown in FIGS. 5D to 5F, the lesion sites of the treatment group showed generally complete structural continuity, suggesting the inventive drug regenerated the nerve cells and had a substantial therapeutic effect on spinal nerve damage. On the other hand, as shown in FIGS. 5A to 5C, the lesion sites of the control group were corroded.

### INDUSTRIAL APPLICABILITY

As described above, the present invention provides the pharmaceutical composition for the treatment of nerve damage, which contains a therapeutically effective amount of blood plasma or serum. The pharmaceutical composition containing blood plasma or serum according to the present invention has the effects of regenerating and healing damaged nerve cells, such that the nerve cells normally function.

Although a specific embodiment of the present invention has been described in detail, those skilled in the art will appreciate that this description is merely a preferred embodiment and is not construed to limit the scope of the present invention.

1. A pharmaceutical composition for the treatment of nerve damage, which contains a pharmaceutically effective amount of blood plasma or serum as an active ingredient.

2. The pharmaceutical composition according to claim 1, which is in the form of any one selected from the group consisting of paste, solution, suspension, and gel for local administration.

3. The pharmaceutical composition according to claim 1, wherein the content of said blood plasma or serum is 0.1-99.9 wt % based on the weight of the composition.

4. The pharmaceutical composition according to claim 1, wherein the nerve damage is peripheral nerve damage or spinal damage.

5. The pharmaceutical composition according to claim 1, wherein the nerve damage is damage in which the nerves are severed.

6. The pharmaceutical composition according to claim 5, wherein the damage, in which the nerves are severed, is damage in which the length of severed neurites are longer than 10 mm.

7. The pharmaceutical composition according to claim 1, wherein the peripheral nerve damage is a nerve disorder associated with a disease selected from the group consisting of diabetic neuropathy, acromegaly, hypothyroidism, AIDS, leprosy, Lyme disease, systemic lupus erythematosus, rheumatoid arthritis, Sjogren's syndrome, periarteritis nodosa, Wegener's granulomatosis, cranial arteritis and sarcoidosis.

8. A method of treating nerve damage, comprising use of a pharmaceutically effective amount of blood plasma or serum for said treatment.
